(19) United States
(12) Patent Application Publication
(10) Pub. No.: US 2009/0086160 A1
(43) Pub. Date: Apr. 2, 2009

(54) METHODS OF STERILIZING OPHTHALMIC LENSES

(76) Inventors: John B. Enns, Jacksonville, FL (US); Venkatesh Ketty, Jacksonville, FL (US); John Frost, Jacksonville, FL (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/238,583

(22) Filed: Sep. 26, 2008

Related U.S. Application Data
(60) Provisional application No. 61/011,511, filed on Sep. 28, 2007.

(51) Int. Cl. G02C 7/02; A61L 2/10

(52) U.S. Cl. 351/159; 422/24

(57) ABSTRACT

Methods of sterilizing ophthalmic lenses are disclosed.

### RELATED APPLICATIONS

This application is a non-provisional filing of a provisional application U.S. Pat. App. No. 61/011,511.

### BACKGROUND

Contact lenses have been available commercially since the 1950s to improve vision. The first contact lenses were made of hard materials and later developments in the field produced soft hydrogel lenses and silicone hydrogel lenses. As a product that is designed to be inserted into the eye of a patient, ophthalmic lenses must be sterilized during their manufacturing process. Initially, ophthalmic lenses were sterilized by steam sterilization. In this process, ophthalmic lenses that are immersed in packaging solution are hermetically sealed and heated to a particular temperature for a period of time. The lenses, which were manufactured and placed into their package along with a packing solution, were manually removed from the manufacturing line by an operator and placed in a steam sterilizer that was not connected to the manufacturing line. Later developments resulted in a fully automated processes that did not require an operator to manually remove the lenses from the manufacturing line. Even though this was an advance, the basic process of heating the sealed ophthalmic lenses for a period of time, typically heating at a temperature of about 120 to 124° C. for about 18-24 minutes, is still practiced. It would be advantageous to the art if other methods of sterilizing ophthalmic lenses were discovered. The following invention meets this need.

### BRIEF DESCRIPTION OF THE DRAWINGS

FIG. 1 Comparison of sterilization rates using methods of the invention.

### DETAILED DESCRIPTION OF THE INVENTION

This invention includes a method of sterilizing an ophthalmic lens that is hermetically sealed in its primary packaging comprising agitating said ophthalmic lens and its primary packaging and irradiating said ophthalmic lens and its primary packaging with a monochromatic UV light source having an intensity of about 10 mW/cm².

As used herein “ophthalmic lens” refers to an ophthalmic device that resides in or on the eye. These devices can provide optical correction or may be cosmetic. The term lens includes but is not limited to hard contact lenses and soft contact lenses, intraocular lenses, overlay lenses, ocular inserts, and optical inserts. The preferred lenses of the invention are soft contact lenses are made from silicone elastomers or hydrogels, which include but are not limited to silicone hydrogels, and fluorohydrogels. Soft contact lens formulations are disclosed in U.S. Pat. No. 5,710,302, WO 9421698, EP 406161, JP 2000016905, U.S. Pat. No. 5,998,498, U.S. patent application Ser. No. 09/532,943, U.S. Pat. No. 6,087,415, U.S. Pat. No. 5,760,100, U.S. Pat. No. 5,776,999, U.S. Pat. No. 5,789,461, U.S. Pat. No. 5,849,811, and U.S. Pat. No. 5,965,631. The foregoing references are hereby incorporated by reference in their entirety. The particularly preferred lenses of the inventions made from etafilcon A, genfilcon A, galyfilcon A, senofilcon A, lenefilcon A, narafilcon A, lotrafilcon A, lotrafilcon B, balifilcon A, or polymacon. More particularly preferred lenses of the invention made from genfilcon A, galyfilcon A, senofilcon A, lenefilcon A, narafilcon A, lotrafilcon A, lotrafilcon B, or balifilcon A,. The most preferred lenses include but are not limited to galyfilcon A, senofilcon A, narafilcon A, and lenses disclosed in U.S. Pat. App. No. 60/318,536, entitled Biomedical Devices Containing Internal wetting Agents,” filed on Sep. 10, 2001 and its non-provisional counterpart of the same title, U.S. Ser. No. 10/236,538, filed on Sep. 6, 2002, U.S. Pat. No. 6,087,415, U.S. Pat. No. 5,760,100, U.S. Pat. No. 5,776,999, U.S. Pat. No. 5,789,461, U.S. Pat. No. 5,849,811, and U.S. Pat. No. 5,965,631. These patents as well as all other patents disclosed in this application are hereby incorporated by reference in their entirety.

As used herein, primary packaging refers to a single lens storage unit commonly known as a blister package. Typical blister packages have a portion that houses the lens with or without packaging solution and a cover that is hermetically sealed to the lens housing portion. Examples of such primary packages include but are not limited to those disclosed in the following publications, U.S. Pat. Nos. D 435,966 S; 4,691,820; 5,467,868; 5,704,468; 5,823,327; 6,050,398, which are hereby incorporated by reference in their entirety. Typically the cover portion is flexible material such an aluminum laminate, however it is preferred that the cover is a transparent to the wavelength of the sterilizing radiation material such as a laminate of a variety of different polymers. The preferred housing and cover portions transmit UV light through those materials to the ophthalmic lens enclosed in the primary package. It is preferred that the primary package transmit at least about 10% to about 100% of the monochromatic UV light that irradiates the primary package. The cover and the housing portion of the primary package are hermetically sealed by a number of methods, preferably by heat sealing.

As used herein “agitating,” means shaking, rotating or otherwise moving the package during irradiation of the lens with a monochromatic light source. It is preferred that the primary package be agitated during irradiation. It is particularly preferred that the package be shaken.

Monochromatic light sources include but are not limited to excimer lamps of a particular intensity. One or more of such light sources may be used to increase the intensity of light that the primary packaging and the ophthalmic lens receive. It is preferred that the primary packaging and the ophthalmic lens are exposed to monochromatic UV lights having an intensity of about 10 mW/cm² to about 1000 mW/cm², more preferably about 50 mW/cm² to about 300 mW/cm². The preferred wavelength of the monochromatic UV light is about 282±10 nm.

As used herein “packaging solutions” of the invention may be water-based solutions. Typical solutions include, without limitation, saline solutions, other buffered solutions, and deionized water. The preferred aqueous solution is deionized water or saline solution containing salts including, without limitation, sodium chloride, sodium borate, sodium phosphate, sodium hydrogenphosphate, sodium dihydrogenphosphate, or the corresponding potassium salts of the same. These ingredients are generally combined to form buffered solutions that include an acid and its conjugate base, so that addition of acids and bases cause only a relatively small change in pH. The buffered solutions may additionally include 2-(N-morpholino)ethanesulfonic acid (MES), sodium hydroxide, 2,2-bis(hydroxymethyl)-2,2′,2″-nitrilotriethanol, n-tris(hydroxymethyl)methyl-2-aminoethanesulfonic acid, citric acid, sodium citrate, sodium carbonate, sodium bicarbonate, acetic acid, sodium acetate, ethylenediamine tetraacetic acid and the like and combinations thereof. Preferably, the solution is a borate buffered or phosphate buffered saline solution or deionized water.

In addition the invention includes an ophthalmic lens that is hermetically sealed in its primary packaging made by the method of agitating said ophthalmic lens and its primary packaging and irradiating said ophthalmic lens and its primary packaging with a monochromatic UV light source having an intensity of about 10 mW/cm².

### EXAMPLES

### Example 1

Senofilcon A lenses were prepared using known methods and placed into polypropylene blister packages with 900 μL of a borate buffered saline packaging. Each of the packages was inoculated with Bacillus pumilus prepared to 10⁶ spores/mL. The packages were heat sealed with a transparent lidstock. One set of packages was placed in the radiation chamber and agitated (shaken back and forth at 15 Hz with an amplitude of 0.375 inches) while they were exposed to the radiation, and the other set of packages was placed in the radiation chamber and exposed without the benefit of agitation. Each set was irradiated with two excimer lamps (one above the package and one below the package) and total intensity of radiation to reach the package is 115 mW/cm². The packages were irradiated for between 4.3 seconds and 87 seconds to give a particular dose between 0.5 and 10 J/cm² of exposure. After exposure the packages were assayed to determine the extent of microbial inactivation and listed as number of samples with microbial activity vs. total number of packages tested. The data are listed in Table 1. This data shows that at tested dose levels, shaking the package reduces or eliminates microbial activity in treated packages.

TABLE 1
Number of packages exhibiting microbial activity after exposure

| Dose | | |
|---|---|---|
| (J/cm²⁾ | No shaking | Shaking |
| 0.5 | 37/100 | 1/100 |
| 2.0 | 15/100 | 1/100 |
| 8.0 | 3/100 | 0/100 |
| 10.0 | 1/100 | 0/100 |

### Example 2

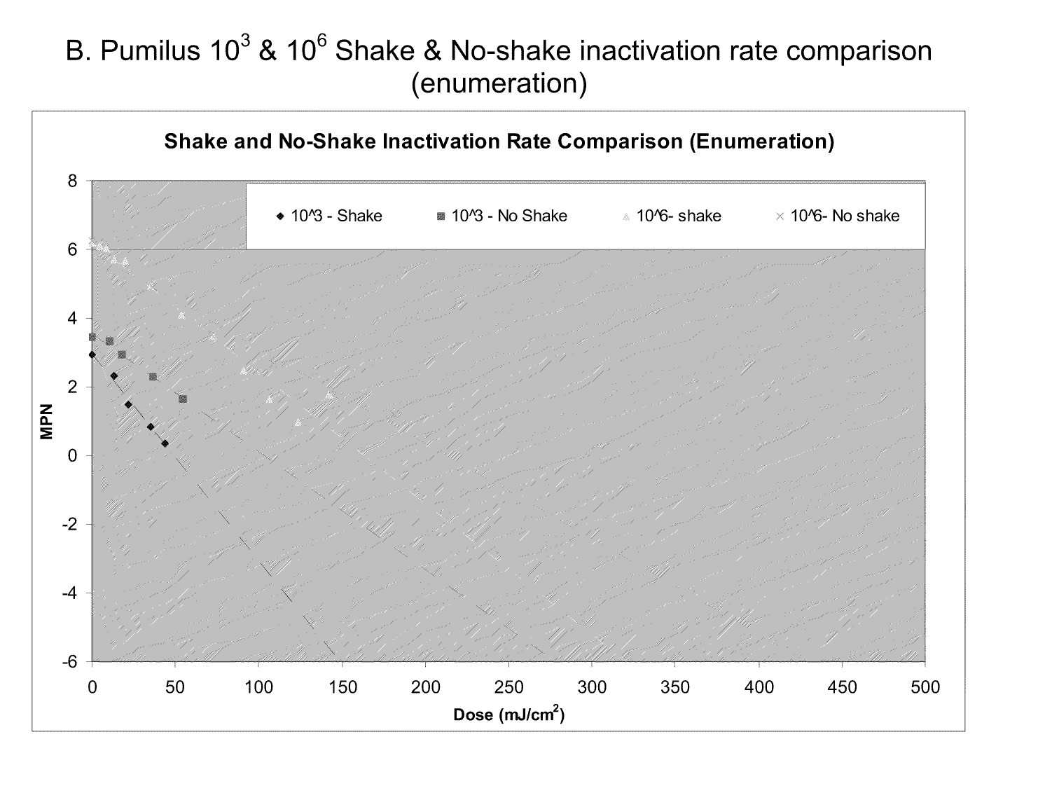

Senofilcon A lenses were prepared, packaged, and sealed as in Example 1, except that some packages were inoculated with Bacillus pumilus prepare to 10⁶ spores/mL and others were inoculated with Bacillus pumilus to prepare 10³ spores/mL. A portion of the packages of each inoculation were shaken while they were irradiated as in Example 1. The packages were assayed to determine the bacterial count. The data are presented in FIG. 1. This figure illustrates that shaking reduces the dosage (mJ/cm²) required to sterilize the packages.

What is claimed is:

1. A method of sterilizing an ophthalmic lens that is hermetically sealed in its primary packaging comprising agitating said ophthalmic lens and its primary packaging and irradiating said ophthalmic lens and its primary packaging with a monochromatic UV light source having an intensity of about 10 mW/cm 2.

2. The method of claim 1 wherein the intensity of the light source is about 10 mW/cm² to about 1000 mW/cm².

3. The method of claim 1 wherein the intensity of the light source is about 50 mW/cm² to about 300 mW/cm².

4. The method of claim 1 wherein the wavelength of light source is about 282±10 nm.

5. The method of claim 1 wherein the ophthalmic lens, and its primary packaging further comprises a packaging solution.

6. The method of claim 1 wherein the ophthalmic lens is selected from the group consisting of etafilcon A, genfilcon A, galyfilcon A, senofilcon A, lenefilcon A, lotrfilcon A, lotrifilcon B, balifilcon A, and polymacon.

7. An ophthalmic lens that is hermetically sealed in its primary packaging made by the method of agitating said ophthalmic lens and its primary packaging and irradiating said ophthalmic lens and its primary packaging with a monochromatic UV light source having an intensity of about 10 mW/cm².

8. The ophthalmic lens of claims 7 wherein the intensity of the light source is about 10 mW/cm² to about 1000 mW/cm².

9. The ophthalmic lens of claims 7 wherein the intensity of the light source is about 50 mW/cm² to about 300 mW/cm².

10. The ophthalmic lens of claims 7 wherein the wavelength of light source is about 282±10 nM.

11. The ophthalmic lens of claims 7 wherein the ophthalmic lens, and its primary packaging further comprises a packaging solution.

12. The ophthalmic lens of claims 7 wherein the ophthalmic lens is selected from the group consisting of etafilcon A, genfilcon A, galifilcon A, senofilcon A, lenefilcon A, lotrfilcon A, lotrifilcon B, balifilcon A, and polymacon.
